(19) United States
(12) Patent Application Publication
(10) Pub. No.: US 2002/0026134 A1
(43) Pub. Date: Feb. 28, 2002

(54) Disposable cover for a lifting sling

(76) Inventors: Ian David Dalton, Gloucestershire (GB); Cho Kee Wong, Kowloon (HK)

Correspondence Address: YOUNG & THOMPSON, 745 SOUTH 23RD STREET 2ND FLOOR, ARLINGTON, VA, 22202

(21) Appl. No.: 09/899,924

(22) Filed: Jul. 9, 2001

(30) Foreign Application Priority Data
Jul. 8, 2000 (GB) 0016754.4
Jul. 19, 2000 (GB) 0017583.6

(51) Int. Cl. A61F 5/00

(52) U.S. Cl. 602/3

(57) ABSTRACT

The disposable cover comprises two sheets of non-woven impermeable material secured together along first edges to define therebetween a pocket for receiving a lifting sling. A tear line may be provided at or adjacent to at least one of said first edges to facilitate removal of the cover from the sling.

### INTRODUCTION

This invention relates to a cover for a lifting sling and to a method of preventing cross-infection between patients lifted in body support slings particularly, but not exclusively, one-piece lifting slings. Such slings support the back and thighs of a patient, being suspended from a hoist by detachable suspension means, such as straps or the like.

Most known slings are formed from woven, synthetic textile material and are relatively expensive to make. The slings are used, inter alia, to support patients, e.g. to the toilet. Accidents do occur and cross-infection between patients has become a major issue. As a result, some hospitals have banned the use of slings.

Laundering does not always kill off the organisms which cause infections, particularly when laundering at temperatures which the slings will withstand. As a result, slings have been laundered and, more particularly, dried at temperatures greater than the slings will withstand in an attempt to kill off all infectious organisms and this has resulted in the destruction of slings.

### SUMMARY OF THE INVENTION

According to a first aspect of the present invention, there is provided a disposable cover for a lifting sling comprising two sheets (or sheet portions) of non-woven impermeable material secured together along first edges to define therebetween a pocket for receiving a lifting sling.

Preferred and/or optional features of the first aspect of the invention are set out in claims 1 to 7, inclusive.

According to a second aspect of the invention, there is provided a method of preventing cross-infection between patients lifted in body support slings suspended from a lifting hoist, the method comprising the steps of:

a. providing a disposable cover on the sling.

b. lifting the patient in the sling, and

c. disposing of the cover prior to lifting another patient with the sling.

Preferred and/or optional features of the second aspect of the invention are set out in claims 10 and 11.

It has been found that these disposable sling covers can be made at a fraction of the cost of slings of woven material. It is, therefore, possible to dedicate the covers to a single toileting procedure and to dispose of the covers thereafter.

The invention will now be more particularly described, by way of example, with reference to the accompanying drawings.

### BRIEF DESCRIPTION OF THE DRAWINGS

FIGS. 1 and 2 are perspective views illustrating one embodiment of a sling cover according to the invention.

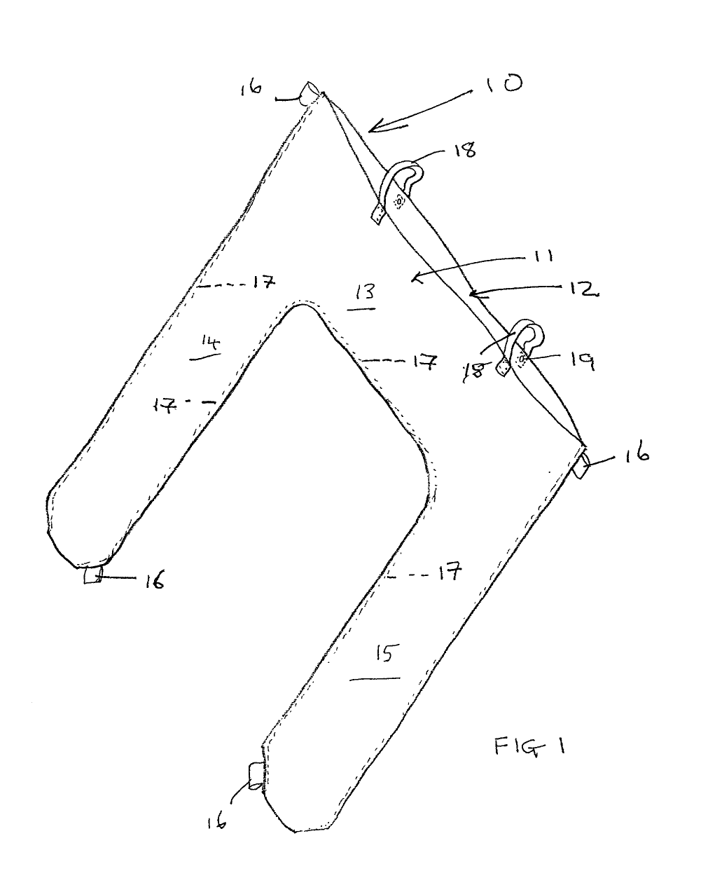

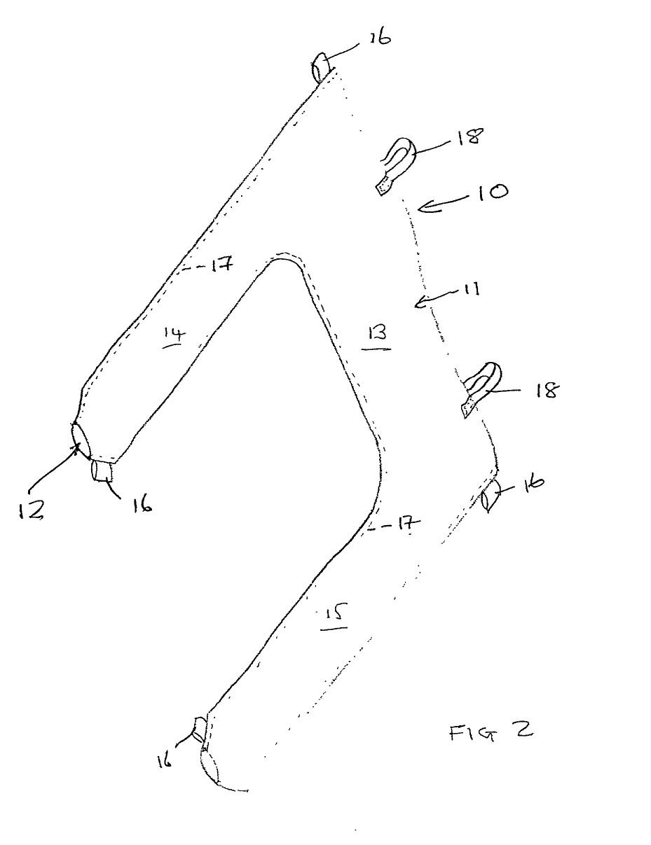

### DESCRIPTION OF THE PREFERRED EMBODIMENT

Referring to the drawings, the disposable sling cover 10 shown therein is formed of two sheets 11,12 of non-woven impermeable material, such as spun bond polyester. Each sheet 11,12 comprises a main portion 13 for receiving a main portion of a sling which portion in use supports the lower back of a patient and lower end dependent leg portions 14 and 15 for receiving lower end dependent leg portions of a sling which portions in use, respectively, extend beneath and upwardly between the thighs of a patient.

The two sheets 11, 12 are secured together, such as by stitching, along outer side edges of the main portion 13, along the lowermost edge of the main portion 13 between the dependent leg portions 14 and 15 and along the outer and inner edges of the lower end dependent leg portions 14 and 15. The two sheets 11, 12 are not secured together along the uppermost edge of the main portion 13 so as to allow a sling to be inserted into the pocket between the two sheets and they are not secured together at the lowermost ends of the lower end dependent leg portions 14 and 15 so as to enable suspension tapes secured to the sling to pass therethrough. Tabs 16 are also secured between the two sheets 11, 12, such as by the previous stitching operation, for a purpose which will become apparent hereinafter.

Tear lines 17 are provided at or adjacent to each of the edges where the two sheets 11 and 12 are secured together. These tear lines can be provided by a regularly spaced weakening of the material of the two sheets 11, 12, such as may be achieved by an ultrasonic heating technique. These tear lines 17 enable the two sheets 11, 12 to be separated by gripping the tabs 16 and tearing the cover along the tear lines 17.

Straps 18 are secured to the sheet 11 adjacent to the uppermost edge of the main portion 13 and are connectible to pop fasteners 19 adjacent to the uppermost edge of the main portion 13 of sheet 12 to releasably fasten the cover to a sling.

In use, prior to lifting a patient in a lifting sling, a cover 10 as described above is provided on the sling. The cover 10 is disposed of prior to lifting another patient with the sling. In particular, the cover 10 is most useful when toileting a patient and the cover can be disposed of after each toileting procedure. Indeed, after a toileting procedure, the lower sheet of the cover can be separated from the upper sheet by tearing along the tear lines 17 and the lower sheet can be disposed of instantly. The upper sheet can then be disposed of after the patient has been lowered and removed from the sling.

Sling covers as described above can be made at a fraction of the cost of woven slings.

Ideally, it should not be possible to launder the sling covers. This will avoid re-use. To this end, it is envisaged that the edges may be secured together by a soluble thread so that the slings will fall apart if laundering is attempted.

The invention is not limited to covers for one-piece lifting slings, but may also be applied to other lifting slings. Also, tear lines may be provided at or adjacent to just some of the secured edges. This will facilitate removal of the cover from the sling, although not allow the two sheets to be separated from one another.

The cover could be formed from two portions of a single sheet integral with one another along one edge and folded about said one edge.

What is claimed is:

1. A disposable cover for a lifting sling comprising two sheets (or sheet portions) of non-woven impermeable material secured together along first edges to define therebetween a pocket for receiving a lifting sling.

2. A disposable cover as claimed in claim 1, wherein a tear line is provided at or adjacent to at least one of said first edges to facilitate removal of the cover from the sling.

3. A disposable cover as claimed in claim 2, wherein tear lines are provided at or adjacent to all of said first edges so that the two sheets (or sheet portions) of material can be separated from one another.

4. A disposable cover as claimed in claim 2, wherein one or more tabs is/are secured to said at least one first edge, the tab or tabs being manually grippable to enable the cover to be torn along the tear line or lines.

5. A disposable cover as claimed in claim 1, wherein the cover is shaped to receive a one piece body support sling which will support the back and thighs of a patient.

6. A disposable cover as claimed in claim 5, wherein the cover includes a main portion for receiving a main portion of a sling which portion in use supports the back of a patient and lower end dependent leg portions for receiving lower end dependent leg portions of a sling which portions in use respectively extend beneath and upwardly between the thighs of a patient.

7. A disposable cover as claimed in claim 1, wherein releasable fastening means are provided along at least one second edge of the cover for fastening the cover to a sling.

8. A method of preventing cross-infection between patients lifted in body support slings suspended from a lifting hoist, the method comprising the steps of:
a. providing a disposable cover on the sling,
b. lifting the patient in the sling, and
c. disposing of the cover prior to lifting another patient with the sling.

9. A method as claimed in claim 8, wherein step (b) comprises toileting the patient and wherein the cover is disposed of after each toileting procedure.

10. A method as claimed in claim 8, using a disposable sling cover for a lifting sling comprising two sheets (or sheet portions) of non-woven impermeable material secured together along first edges to define therebetween a pocket for receiving a lifting sling.
